(19) United States
(12) Patent Application Publication
(10) Pub. No.: US 2003/0008855 A1
(43) Pub. Date: Jan. 9, 2003

(54) Anti-dandruff hair styling composition

(76) Inventors: Deanna F. Simon, Lawndale, CA (US); Karen A. Hohenstein, Los Angeles, CA (US); George Andrassy, Walnut, CA (US)

Correspondence Address: HENKEL CORPORATION, 2500 RENAISSANCE BLVD, STE 200, GULPH MILLS, PA, 19406, US

(21) Appl. No.: 09/932,770

(22) Filed: Aug. 17, 2001

Related U.S. Application Data
(60) Provisional application No. 60/300,499, filed on Jun. 22, 2001.

(51) Int. Cl. A61K 31/555; A61K 7/06; A61K 7/11

(52) U.S. Cl. 514/184; 424/70.13; 424/70.16

(57) ABSTRACT

The present invention provides a hair styling composition containing at least one active ingredient for the treatment of dandruff and/or seborrheic dermatitis; at least one thickening agent; and at least one a hair styling resin. In a preferred embodiment of the present invention, the hair styling composition is in the form of a gel that suspends the active ingredient. The present invention also provides a method of treating hair or scalp using the composition of the present invention.

### CROSS REFERENCE TO RELATED APPLICATIONS

This application claims the benefit under 35 U.S.C. §119(e) to U.S. provisional application Serial No. 60/300,499 filed on Jun. 22, 2001, the disclosure of which is hereby incorporated by reference in its entirety.

### FIELD OF THE INVENTION

The present invention relates to a hair styling composition and to a method of treating the scalp and/or hair. The composition treats dandruff and/or seborrheic dermatitis while aiding in styling the hair or maintaining a hair style.

### BACKGROUND OF THE INVENTION

Actives useful for the treatment of dandruff or seborrheic dermatitis include for example metal salts of pyridinethione, such as the zinc or sodium salts of 1-hydroxy-2-pyridinethione or 2-pyridinethiol-1-oxide; octopirox (hydroxy-4-methyl-6-(2,4,4-trimethylpentyl)-2-pyridone-monoethanolamine salt or INCI name: Piroctone Olamine); selenium disulfide, or combinations thereof. These actives are most typically added to anti-dandruff shampoos which are designed to treat the symptoms of dandruff while effectively cleaning the hair. Much research has been directed to the development of these shampoos, and many patents have been issued in this area. For example, U.S. Pat. No. 5,104,645 to Cardin et al., discloses a shampoo formulation containing metal pyridinethione salts of a particular platelet size in combination with a synergizer compound. U.S. Pat. No. 4,033,895 to Gerstein discloses a shampoo formulation containing stearylamine oxide in combination with sodium lauryl sulfate and zinc pyridinethione to reduce the irritant properties of the shampoo. Several drawbacks of using shampoo compositions to deliver these active ingredients include the short contact time of the shampoo with the scalp, and the potential irritation of the scalp by the shampoo formulation.

Hair styling compositions are applied to the hair and commonly used to assist one in obtaining a particular hair style or shape and/or to retain a particular hair style or shape. Examples of hair styling compositions include gels, mousses, lotions, or sprays (including sprayable gels). It would be desirable to provide one or more anti-dandruff agents in a leave-on hair styling composition to provide a more gentle and efficacious way to treat the scalp for dandruff and/or seborrheic dermatitis. For example, harsh surfactants used in shampoos can be eliminated, and conditioning and soothing ingredients can easily be incorporated into the hair styling composition if desired.

U.S. Pat. No. 3,636,213 discloses a method for solubilizing heavy metal salts of 1-hydroxy-2-pyridinethione. The method includes admixing the heavy metal salts of pyridinethione with an amine or polyalkylene imine to achieve solubilization of the pyridinethione. The method is taught as being useful for such compositions as hair rinses, shampoos, alcoholic hair dressings, hair setting lotions and disinfectant solutions. Unfortunately, this method introduces additional ingredients for solubilizing the active ingredient that may be irritating to the scalp and skin due to the pH. Additionally, one is limited in the amount of active ingredient that can be added due to solubility limitations of the active ingredient.

The present invention provides a hair styling composition that is preferably in the form of a gel and contains an active ingredient for the treatment of dandruff and/or seborrheic dermatitis. The hair styling composition is preferably designed to suspend the active ingredient in the composition for effective delivery of the active ingredient to the hair and/or scalp.

### SUMMARY OF THE INVENTION

The present invention provides a hair styling composition containing at least one active ingredient for the treatment of dandruff or seborrheic dermatitis that includes a pyridinethione metal salt, octopirox, selenium disulfide, or combinations thereof. The hair styling composition also contains at least about 0.01 weight percent of at least one polymeric thickening agent; and at least about 0.10 weight percent of at least one hair styling resin, where the composition has a viscosity of at least about 4,000 centipoises. The thickening agent includes a hydroxy C₁ to C₆ alkyl ether polyaldose, a copolymer having polymerized units of at least one carboxylic acid containing monomer and at least one hydrophobic monomer, or combinations thereof.

The present invention also provides a method for treating a scalp or hair that includes applying the hair styling composition of the present invention to the scalp or the hair where the composition treats the scalp or hair for dandruff or seborrheic dermatitis and is capable of assisting in styling the hair or maintaining the hair in a hair style.

### DETAILED DESCRIPTION OF THE INVENTION

The hair styling composition of the present invention contains a) at least one active ingredient for the treatment of dandruff, seborrheic dermatitis, or combinations thereof; b) at least one thickening agent for at least partially suspending the active ingredient; and c) at least one hair styling resin. The hair styling composition may also contain other ingredients to form a cosmetically acceptable formulation for application to the hair such as water, ethanol, and/or pH adjusters.

“(Meth)” when used in the names of monomers herein, such as “(meth)acrylic acid” or “(meth)acryloyl” methyl betaine, means that the “meth” group is optional in the monomer. For example, “(meth)acrylic acid” means methacrylic acid or acrylic acid. “Salts thereof” means the salt product formed when an acid functional group or basic functional group located on a compound (e.g., monomer or polymer), is reacted, respectively, with an organic or inorganic base or an organic or inorganic acid. For example, acrylic acid can be neutralized with sodium hydroxide to form sodium acrylate. The resulting salt should be cosmetically compatible in the hair styling composition. By “cosmetically compatible,” it is meant that the composition is suitable for application to human hair, including the scalp and skin without undue toxicity, instability, allergic response, and/or irritation.

By “hair styling composition,” as used herein, it is meant a formulation applied to the hair that aids in styling the hair, such as shaping the hair, or aids in retaining a particular hair style. The hair styling composition may be in the form, for example, of a lotion, gel, sprayable gel, mousse or spray. Preferably, the hair styling composition is in the form of a gel. By “gel,” as used herein, it is meant a formulation that can be clear, semi-opaque, or opaque, and that has a viscosity between about 4,000 centipoises and about 100,000 centipoises.

The hair styling composition preferably has a viscosity that will at least partially suspend, and more preferably completely suspend the active ingredient used for treatment of dandruff and/or seborrheic dermatitis. The hair styling composition preferably has a viscosity of at least about 4,000 centipoises, more preferably from about 10,000 centipoises to about 100,000 centipoises, and most preferably from about 15,000 centipoises to about 35,000 centipoises. Viscosity measurements, as provided herein, are preferably obtained at a temperature of 25° C., using a Brookfield viscometer, spindle no. 6 at 20 revolutions per minute (rpm).

As previously described, the hair styling composition of the present invention contains at least one active ingredient for treatment of dandruff and/or seborrheic dermatitis. Any active ingredient useful for the treatment of dandruff or seborrheic dermatitis may be used in the hair styling composition of the present invention. Preferred active ingredients include, for example, the metal salts of pyridinethione, such as the salts of 1-hydroxy-2-pyridinethione or 2-pyridinethiol-1-oxide; octopirox (hydroxy-4-methyl-6 (2,4,4-trimethylpentyl)-2-pyridone-monoethanolamine salt or INCI name: Piroctone Olamine); selenium disulfide, or combinations thereof. More preferably, the active ingredient is a water-insoluble metal salt of pyridinethione. By “water-insoluble” it is meant that the active ingredient has a solubility in water of less than about 0.001 grams per liter at 25° C. Such compounds include for example zinc, tin, cadmium, magnesium, aluminum, sodium, or zirconium salts of pyridinethione. Preferably, the pyridinethione compound is a salt of zinc and/or sodium, and most preferably zinc (i.e., zinc pyrithione).

The active ingredient may be present in the composition at any level that effectively treats dandruff or seborrheic dermatitis. Preferably, the active ingredient is present at a level that complies with a country's regulations for using the active ingredient. For example, if used in the United States, preferably the active ingredient level complies with the United States Code of Federal Regulations Monograph for Over the Counter Drug Products for the Treatment of Dandruff and Seborrheic Dermatitis. Generally, the active ingredient is preferably present at a level of at least about 0.05 weight percent, more preferably from about 0.07 weight percent to about 0.30 weight percent, and most preferably from about 0.1 weight percent to about 0.25 weight percent, based on the total weight of the composition.

The hair styling composition also contains at least one thickening agent to at least partially suspend, and preferably completely suspend the active ingredient for treating dandruff or seborrheic dermatitis. By “thickening agent,” as used herein, it is meant any substance, which may be natural, synthetic, or semi-synthetic, that increases the viscosity of the hair styling composition, and includes, for example, gelling agents that form gels (i.e., gellants). The thickening agent, in addition to suspending the active ingredient for treating dandruff or seborrheic dermatitis, must also be compatible with the active ingredient so that, for example, the thickening agent or active ingredient does not precipitate from the composition. Additionally, the thickening agent should not breakdown in viscosity in the presence of the active ingredient. It was surprisingly discovered that many thickening agents traditionally used in hair styling compositions were incompatible with the active ingredient.

Thickening agents useful in the present invention include polymers that provide thickening (an increase in viscosity) in an aqueous-based system. By “aqueous-based system” it is meant a composition containing at least about 70 weight percent water, based on the total weight of the composition. These polymers are preferably water-soluble or water-swellable. By “water soluble or water swellable” it is meant that the thickening agent is soluble and/or swells in an aqueous-based system at the use concentration of the thickening agent in the aqueous-based system, and does not precipitate out of the system. It has been discovered that thickening agents particularly useful in the present invention include certain polysaccharides having a weight average molecular of at least about 80,000 daltons and certain acid containing copolymers having a weight average molecular weight of at least about 90,000 daltons.

The polysaccharides useful in the present invention as thickening agents include nonionic hydroxy C₁ to C₆ alkyl ether polyaldoses. By “polyaldose”, it is meant a polymer containing aldose units such as pentose (ribose, arabinose, xylose or apiose) or hexose (glucose, fucose, mannose, or galactose) or combinations thereof. Preferably, the polyaldose has a weight average molecular weight of at least about 80,000 daltons, more preferably from about 500,000 daltons to about 2,000,000 daltons and most preferably from about 800,000 daltons to about 1,600,000 daltons. The weight percentage of aldose units, based on the total weight of the polyaldose, is at least about 50 weight percent, more preferably at least about 75 weight percent and most preferably from about 85 weight percent to 100 weight percent.

Preferred polyaldoses include for example hydroxy C₁ to C₆ alkyl celluloses, such as hydroxymethylcellulose, hydroxyethylcellulose, hydroxypropyl cellulose, hydroxy methylpropylcellulose or hydroxybutylcellulose; or hydroxy C₁ to C₆ alkyl guars, such as hydroxyethyl guar, hydroxypropyl guar, or hydroxybutyl guar, or combinations thereof.

The thickening agent useful in the present invention may also be a copolymer containing polymerized units of carboxylic acid containing monomer and hydrophobic monomer. By “hydrophobic monomer” it is meant a monomer having solubility in water of about 200 millimoles per liter or less at a temperature of from about 25° C. to about 50° C. Preferably, the copolymer has a weight average molecular weight of at least about 90,000 daltons and more preferably from about 100,000 daltons to about 5,000,000 daltons. In a preferred embodiment, the copolymer is crosslinked.

Preferably, the amount of carboxylic acid containing monomer in the copolymer is enough to impart water solubility or water swellability to the copolymer. Preferably, the amount of carboxylic acid containing monomer is from about 5 weight percent to about 99 weight percent, and more preferably from about 20 weight percent to about 95 weight percent, based on the total weight of the copolymer. The amount of hydrophobic monomer present in the copolymer is preferably from about 1 weight percent to about 95 weight percent, and more preferably from about 5 weight percent to about 80 weight percent, based on the total weight of the copolymer.

Carboxylic acid containing monomers that may be present in the copolymer thickening agent include, for example, monoethylenically unsaturated monomers containing at least one carboxylic acid group such as (meth)acrylic acid, (meth)acryloxypropionic acid, itaconic acid, maleic acid, maleic anhydride, fumaric acid, crotonic acid, monoalkyl maleates, monoalkyl fumarates, monoalkyl itaconates, salts of the foregoing monomers, or combinations thereof. Preferred carboxylic acid containing monomers include (meth)acrylic acid, crotonic acid, maleic acid, maleic anhydride, salts of the foregoing monomers, or combinations thereof.

Hydrophobic monomers that may be present in the copolymer thickening agent include, for example, α-, β-, ethylenically unsaturated hydrophobic monomers such as vinyl esters of C₆ to C₃₀ carboxylic acids, such as vinyl hexanoate, vinyl heptanoate, vinyl 2-ethylhexanoate, vinyl octanoate, vinyl nonanoate, vinyl decanoate, vinyl isodecanoate, vinyl neodecanoate, vinyl laurate, or vinyl stearate; primary alkenes; styrene or alkylsubstituted styrene; a-methyl styrene; vinyltoluene; vinyl chloride; vinylidene chloride; N-alkyl substituted (meth)acrylamide such as octyl acrylamide or maleic acid amide; vinyl alkyl or aryl ethers with (C₁-C₃₀) alkyl groups such as methyl vinyl ether, ethyl vinyl ether, propyl vinyl ether, stearyl vinyl ether; or (C₁-C₃₀) alkyl esters of (meth)acrylic acid, such as methyl methacrylate, ethyl (meth)acrylate, butyl (meth)acrylate, 2-ethylhexyl (meth)acrylate, benzyl (meth)acrylate, lauryl (meth)acrylate, oleyl (meth)acrylate, palmityl (meth)acrylate, stearyl (meth)acrylate; unsaturated vinyl esters of (meth)acrylic acid such as those derived from fatty acids or fatty alcohols; or combinations thereof. Preferred hydrophobic monomers include vinyl esters of C₆ to C₁₈ carboxylic acids; C₁ to C₅ alkyl vinyl ethers; C₁ to C₈ alkyl esters of (meth)acrylic acid; or combinations thereof.

Crosslinking agents that may optionally be used are any of those known in the art. For example, crosslinking agents disclosed in U.S. Pat. No. 5,032,391 may be used. Crosslinking agents used in free radical polymerizations are especially preferred.

Preferably, the copolymer is a crosslinked copolymer containing polymerized units of (meth)acrylic acid and a vinyl ester of a C₆ to C₁₈ carboxylic acid; a crosslinked copolymer containing polymerized units of maleic anhydride or maleic acid and a C₁ to C₅ alkyl vinyl ether monomer, such as those polymers described in U.S. Pat. No. 5,032,391; salts of the foregoing polymers, or combinations thereof.

In a preferred embodiment of the present invention, the thickening agent includes a crosslinked copolymer having polymerized units of (meth)acrylic acid and a vinyl ester of a C₆ to C₁₈ carboxylic acid; a crosslinked copolymer containing polymerized units of maleic anhydride or maleic acid and a C₁ to C₂ alkyl vinyl ether monomer; a hydroxy C₂ to C₃ alkyl cellulose, a hydroxy C₂ to C₃ alkyl guar; salts of the foregoing polymers, or combinations thereof. More preferably, the thickening agent includes a crosslinked copolymer having polymerized units of acrylic acid and vinyl isodecanoate such as Acrylates/Vinyl Isodecanoate Crosspolymer (e.g., Stabylen 30, supplied by 3V, Inc.); a copolymer having polymerized units of maleic acid, vinyl methyl ether and decadiene such as PVM/MA Decadiene Crosspolymer (e.g., Stabileze® products supplied by ISP); hydroxyethylcellulose (e.g., Natrosol® products, supplied by the Aqualon Division of Hercules Inc.); hydroxypropylcellulose, (e.g., Klucel® products, supplied by the Aqualon Division of Hercules); hydroxypropyl guar (e.g., Jaguar® HP series -8, -60, -105, -120, supplied by Rhodia); salts of the foregoing polymers, or combinations thereof. In a most preferred embodiment of the present invention, the thickening agent includes a crosslinked copolymer having polymerized units of acrylic acid and vinyl isodecanoate (e.g., Acrylates/Vinyl Isodecanoate Crosspolymer) or salts thereof; hydroxypropylcellulose; or combinations thereof.

Preferably, the thickening agent is present in the hair styling composition in an amount to completely suspend the active ingredient for treating dandruff or seborrheic dermatitis. This is generally an amount to provide the hair styling composition with a viscosity of at least about 4,000 centipoises, more preferably from about 10,000 centipoises to about 100,000 centipoises, and most preferably from about 15,000 centipoises to about 35,000 centipoises. Although the amount added will depend on the thickening agent(s) selected, preferably the amount of thickening agent in the hair styling composition is at least about 0.01 weight percent, more preferably from about 0.01 weight percent to about 3.00 weight percent, and most preferably from about 0.01 weight percent to about 2.00 weight percent, based on the total weight of the composition.

One skilled in the art will also recognize that other thickening agents may also be present in the hair styling composition of the present invention as an optional component provided that they are compatible with the active ingredient for treating dandruff or seborrheic dermatitis. Such other thickening agents include, for example, natural polysaccharides, such as xanthan gum, guar gum, or agar gum; polyethylene glycol monoesters and diesters of fatty acids having a molecular weight of at least about 118; poly(meth)acrylic acid or salts thereof; fatty alcohol ethoxylates. These other thickening agents, if present, are preferably present in an amount of less than about 0.10 weight percent, and more preferably in an amount of less than about 0.01 weight percent, based on the total weight of the composition.

The hair styling composition of the present invention also contains at least one hair styling resin. The hair styling resin may be a natural, synthetic, or semi-synthetic material that is preferably film-forming. The hair styling resin typically aids in obtaining a particular hair style or shape, and/or assists in retaining a particular hair style or hair shape. The amount of hair styling resin present in the composition will depend on such factors as the hair styling resin chosen, the hair type for which the composition is intended, and the degree of “hold” desired.

Generally, the hair styling resin is preferably present in the composition in an amount of at least about 0.1 weight percent, more preferably from about 0.5 weight percent to about 5.00 weight percent, and most preferably from about 0.50 weight percent to about 4.00 weight percent, where the weight percentage of hair styling resin is on a solids basis and based on the total weight of the composition.

Any hair styling resin that provides hair styling or hair shaping benefits may be used. Preferred hair styling resins include nonionic, amphoteric, anionic, cationic, or zwitterionic polymers or combinations thereof. Preferably, the hair styling composition of the present invention contains at least one nonionic hair styling polymer, or at least one amphoteric hair styling polymer, or combinations thereof.

Examples of nonionic hair styling polymers that may be used in the present invention include homopolymers or copolymers containing vinylpyrrolidone such as for example polyvinylpyrrolidone (PVP K-15, K-30, K-60, K-90, K-120, supplied by ISP; Luviskol® K series 12, 17, 30, 60, 80, & 90, supplied by BASF); copolymers containing vinyl pyrrolidone and vinyl acetate (PVP/VA series S-630; 735, 635, 535, 335, 235, supplied by ISP; Luviskol® VA, supplied by BASF); copolymers containing vinyl pyrrolidone and dimethylaminopropylmethacrylamide (Styleze® CC-10, supplied by ISP); copolymers containing vinyl pyrrolidone, vinyl acetate and vinyl propionate (Luviskol VAP 343 E, VAP 343 I, VAP 343 PM, supplied by BASF); copolymers containing vinyl pyrrolidone, and polyethyleneimines; homopolymers or copolymers containing polyvinylcaprolactam (Luviskol Plus, supplied by BASF); Corn Starch Modified (e.g., Amaze, supplied by National Starch); or combinations thereof. Most preferred nonionic polymers are polyvinylpyrrolidone, copolymers of vinyl pyrrolidone and vinyl acetate, or Corn Starch Modified, or combinations thereof.

Examples of amphoteric polymers that may be used in the present invention include copolymers containing a C₅ to C₁₀ alkyl acrylamide and C₁ to C₆ alkylamino C₁ to C₄ alkyl (meth)acrylate such as Octylacrylamide/Acrylates/Butylaminoethyl Methacrylate Copolymer (Amphomer® 28-4910, Amphomer LV-71 284971, Lovocryl®-47 28-4947, supplied by National Starch); copolymers containing polymerized units of (meth)acryloyl C₁ to C₄ alkyl betaine monomer such as Methacryloyl ethyl betaine/methacrylates copolymer (Diaformer® products, supplied by Clariant). A preferred amphoteric polymer is the Methacryloyl ethyl betaine/methacrylates copolymer.

Examples of anionic polymers suitable for use in the present invention are homopolymers and/or copolymers of (meth)acrylic acid or crotonic acid; copolymers of acrylic acid and acrylamide; polyhydroxycarboxylic acids; water-soluble or water-dispersible polyesters; polyurethanes; polyureas; salts of the foregoing polymers, or combinations thereof. Preferred anionic polymers include copolymers of tert-butyl acrylate, ethyl acrylate and methacrylic acid (e.g., Luvimer® 100P supplied by BASF); copolymers of N-tert-butylacrylamide, ethyl acrylate and acrylic acid (Ultrahold® 8, supplied by BASF); copolymers of vinyl acetate, crotonic acid and vinyl propionate (e.g., Luviset® CAP, supplied by BASF); maleic anhydride copolymers, optionally reacted with alcohols, or anionic polysiloxanes; copolymers having carboxyl functional groups and containing vinylpyrrolidone, tert-butyl acrylate and methacrylic acid (e.g., Luviskol® VBM, supplied by BASF); copolymers having vinyl acetate, crotonic acid, and vinyl neodecanoate (Resyn 28-2930, supplied by National Starch); Butyl Ester of PVM/MA (Gantrez® A-425; ES-425; ES-435, supplied by ISP); Isopropyl Ester of PVM/MA Copolymer (Gantrez ES-335, supplied by ISP); Ethyl Ester of PVM/MA (Gantrez ES-225; SP-215—supplier ISP); Acrylates/Acrylamide copolymer (Luvimer® 100P; Lumiver Low VOC, supplied by BASF); Methacrylate Copolymer (Balance 0/55, supplied by National Starch); Vinyl Acetate/Crotonic Acid copolymer (Luviset CA 66, supplied by BASF); Methacrylates/Acrylates copolymer/amine salt (Diahold polymers, supplied by Mitsubishi); VA/Butyl Maleate/Isobomyl Acrylate (Advantage Plus terpolymer, supplied by ISP); Acrylates Copolymer (Amerhold® DR-25, supplied by Amerchol); Acrylates/Hydroxyesteracrylates Copolymer (Acudyne® 255, supplied by Rohm and Haas); PVP/Acrylates copolymer (Luviflex VBM 35, supplied by BASF); Diglycol/CHDM/Isophthalates/SIP Copolymer (Eastman AQ 48, AQ 55, supplied by Eastman Chemicals); Acrylates/Octacrylamide Copolymer (Versatyl-42 or Amphomer HC, supplied by National Starch supplier); TBA/AA copolymer (75/25 supplied by Mitsubishi Chemical Corp.); Acrylates Copolymer (Aculyn® 33, supplied by Rohm and Haas); or combinations thereof.

Examples of cationic polymers that may be used in the present invention include various “Polyquatemium” (INCI name) polymers, such as cationic cellulose derivatives (Polyquatemium 4 or 10, supplied for example by National Starch under the name Celquat® such as Celquat H-100, L200, SC-240C, SC230M); quaternized copolymer containing vinyl pyrrolidone and dimethylaminoethyl methacrylate (Polyquaternium-11, for example supplied by ISP as Gafquat® 734, 755N; or supplied by BASF as Luviquat® PQ 11), quaternized acrylamide copolymer (Polyquaternium 7, for example supplied by Calgon as Merquat® 550), copolymers of vinylpyrrolidone and vinylimidazolium salts (Polyquatemium 16, for example Luviquat® FC 370, FC550, FC905, HM552); Polyquaternium-18 (Mirapol® AZ-1, supplied by Rhodia), Polyquatemium-24 (Quatrisoft Polymer LM-200, supplied by Amerchol); Polyquaternium-28 (Gafquat HS-100, supplied by ISP); or Polyquaternium-46 (Luviquat Hold, supplied by BASF); PVP/Dimethylaminoethylmethacrylate (Copolymer 845, 937, 958, supplied by ISP); copolymer containing vinyl caprolactam, vinyl pyrrolidone and dimethylaminoethyl methacrylate (Gaffix® VC-713, H20LD EP-1, supplied by ISP); Chitosan Lactate (Kytamer® L, supplied by Amerchol); Chitosan PCA (Kytamer PC, supplied by Amerchol); or Chitosan Glycolate (Hydagen™ CMF, CMFP, supplied by Henkel) or combinations thereof. Preferred cationic polymers include Polyquatemium-11, Luviquat Hold, or combinations thereof.

Of the foregoing hair styling resins the more preferred hair styling resins include homopolymers or copolymers of vinyl pyrrolidone, such as for example polyvinyl-pyrrolidone having for example a weight average molecular weight of from about 30,000 to about 1,750,000; copolymers containing vinyl acetate and polyvinylpyrrolidone having for example a weight average molecular weight of from about 25,000 to about 99,000; copolymers containing dimethylaminopropylmethacrylamide and vinyl pyrrolidone (e.g., PVP/DMAPA Acrylates Copolymer, supplied by ISP), quatemized copolymers of vinyl pyrrolidone and dimethylaminoethyl methacrylate (Polyquatemium 11); copolymers containing (meth)acryloyl ethyl betaine monomer, such as Methacryloyl Ethyl Betaine/Methacrylates copolymer (e.g., Diaformer Z-301, supplied by Clariant); homopolymers or copolymers of vinyl caprolactam such as polyvinylcaprolactam having a weight average molecular weight of from about 50,000 to about 150,000; or Corn Starch Modified (e.g., Amaze®, supplied by National Starch); or combinations thereof. More preferably, the hair styling resin includes polyvinylpyrrolidone, polyvinylpyrrolidone/vinyl acetate copolymer, polyvinyl caprolactam, PVP/DMAPA Acrylates Copolymer, Polyquaternium 11, Methacryloyl Ethyl Betaine/Acrylates Copolymer or combinations thereof, and most preferably polyvinyl pyrrolidone and Methacryloyl Ethyl Betaine/Acrylates Copolymer.

The active ingredient for treatment of dandruff and/or seborrheic dermatitis, the thickening agent, and hair styling resin are formulated with a carrier to form the hair styling composition of the present invention. The carrier may be any compound or combination of compounds that provides a cosmetically compatible medium for delivering the active ingredient and hair styling resin to hair. The carrier is preferably present in an amount of at least about 70 weight percent, more preferably from about 75 weight percent to about 99 weight percent, and most preferably from about 80 weight percent to about 99 weight percent, based on the total weight of the composition.

Examples of suitable carriers include water, organic solvents, or combinations thereof. Examples of organic solvents include C₂ to C₅ monoalcohols, such as ethanol, propanol, isopropanol, or tert-butyl alcohol; ethylene glycol; ethylene glycol monomethyl ether; ethylene glycol monoethyl ether; ethylene glycol monobutyl ether; ethylene glycol monoethyl ether acetate; propylene glycol; propylene glycol monomethyl ether; dipropylene glycol monomethyl ether; glycerol; or diethylene glycol; or combinations thereof. Preferably, the hair styling composition is aqueous-based and contains from about 70 weight percent to about 99 weight percent, more preferably from about 75 weight percent to about 99 weight percent, and most preferably from about 80 weight percent to about 99 weight percent water, based on the total weight of the composition. Preferably, the organic solvents in the hair styling composition are less than 20 weight percent, more preferably from 0 to 15 weight percent and most preferably from 0 to 10 weight percent. In a most preferred embodiment of the present invention, the hair styling composition is formulated as a monoalcohol-free composition. In another preferred embodiment, the composition is formulated to contain 6.0 wt % or less of volatile organic compounds (VOC), based on the total weight of the composition, if the composition is a gel to comply with California regulations.

The hair styling composition is preferably formulated to have an aqueous pH of from about 4.00 to about 8.00 and most preferably from about 4.50 to about 6.50. To achieve this pH range, pH adjusters are preferably added to the hair styling composition. Suitable pH adjusters include for example organic amines such as triethanolamine, ethanolamine, or aminiomethylpropanol; alkali metal or alkaline earth metal hydroxides such as sodium hydroxide or potassium hydroxide; ammonium hydroxide; basic amino acids such as arginine or lysine; sodium hydroxymethylglycinate; or combinations thereof. The pH adjusters are added in quantities sufficient to adjust the pH to the desired pH.

In addition to the above-described components, other common cosmetic additives may optionally be included in the composition of the present invention, provided that the properties of the hair styling composition are not adversely affected. Such optional cosmetic additives may be found, for example, in the International Cosmetic Ingredients Dictionary, 7th Edition, 1997, published by the CTFA in Washington D.C. Preferably, these optional components, in total, are present in an amount of from 0 to 20 weight percent, more preferably from about 0.1 to about 10 weight percent and most preferably from about 0.10 to about 2.0 weight percent, based on the total weight of the composition.

Examples of optional additive components include, but are not limited to, one or more plasticizers, conditioners, surfactants, solubilizers, protein hydrolyzates, propellants or combinations thereof.

Plasticizers suitable for use in the present invention include, for example, organic, hydrophilic compounds, preferably containing at least one hydroxyl group, such as glycerine, propylene glycol, sorbitol, dipropylene glycol, or diethylene glycol or combinations thereof. Preferably, plasticizers are added in an amount of from about 0.10 weight percent to about 3.00 weight percent and more preferably no more than about 2.00 weight percent, based on the total weight of the composition.

Examples of conditioners suitable for use in the present invention include compounds that soften, strengthen, add shine and/or detangle the hair, such as phospholipids (e.g., soya lecithin, egg lecithin or kephalins), silicone compounds, proteins, amino acids, or combinations thereof. Suitable silicone compounds include, for example, polyalkylsiloxanes, polyarylsiloxanes, polyarylalkylsiloxanes, polyethersiloxanes or silicone resins. Preferably, the conditioners are present in the hair styling composition in an amount of no more than about 1.00 weight percent, more preferably from about 0.01 weight percent to about 0.70 weight percent and most preferably from about 0.001 weight percent to about 0.50 weight percent, based on the total weight of the composition.

Surfactants, such as those used in shampoos, may also optionally be added to the composition of the present invention. Preferably, surfactants are present in an amount of no more than about 3 weight percent, more preferably in an amount of no more than about 1 weight percent, and most preferably in an amount of no more than 0.5 weight percent, based on the total weight of the composition. Surfactants may include for example, anionic surfactants, such fatty alkyl sulfates or ether sulfates, cationic surfactants, such as for example quaternary ammonium compounds, zwitterionic surfactants, such as betaines, or ampholytic surfactants, or combinations thereof.

The hair styling composition may also contain solubilizers for solubilizing such components as fragrances or oils in an amount of up to about 2.0 weight percent, based on the total weight of the composition. Solubilizers include for example fatty alcohol ethoxylates, sorbitan esters, or combinations thereof.

Protein hydrolyzates may also be present in the composition in an amount of up to about 2.0 weight percent, based on the total weight of the composition. Examples of protein hydrolyzates include elastin, collagen, keratin, milk protein, soya protein, or wheat protein hydrolyzates, or condensation products thereof with fatty acids, or quaternized protein hydrolyzates, or combinations thereof.

Although not as preferred, the hair styling composition may be formulated in a manner that requires a propellant. Suitable propellants include for example butane, mixtures of propane/butane, isobutane, isopentane, nitrous oxide (N₂O), dimethyl ether, carbon dioxide, air, or combinations thereof. If present, the propellant is added to the hair styling composition in an amount of from about 1.0 weight percent to about 10.0 weight percent, based on the total weight of the composition.

The hair styling composition of the present invention may also contain as optional additive components, one or more sequestrants such as ethylene diamine tetraacetic acid (EDTA), or phosphonic acids; fragrances; other nonactive ingredients such as panthenol, allantoin, pyrrolidone carboxylic acids or salts thereof; plant extracts; vitamins such as Vitamins A, B, C, or E, or combinations thereof; preservatives such methylchloroisothiazolinone, methylisothiazolinone, or diazolidinyl urea or combinations thereof; humectants, such as sorbitol, glycerin, propylene glycol, or butylene glycol or combinations thereof; light stabilizers; dyes; pearlescers, such as ethylene glycol monostearate or distearate; antioxidants; or any combination of any of the foregoing additives. The selection of these optional additives will depend upon the desired end use of the hair styling composition. Preferably, these optional additives are present individually in the composition in an amount of no more than about 2.00 weight percent and more preferably in an amount of no more than about 0.50 weight percent.

In another embodiment of the present invention, a method of treating the scalp or hair is provided that includes applying the hair styling composition of the present invention to the scalp or hair to treat dandruff or seborrheic dermatitis; and to style the hair or to maintain the hair in a hair style for a period of time.

The hair styling composition may be applied to wet, semi-dry or dry hair. The amount of hair styling composition applied to the hair is preferably in an amount sufficient to achieve the desired hair style and to effectively deliver the active ingredient for treatment of dandruff or seborrheic dermatitis. For example, although the amount applied will depend on the hair, such as the hair length, thickness of the hair fiber, and density of hair, preferably from about 2.0 grams to about 30.0 grams will be applied. Preferably, the hair styling composition is applied at least once a day, and preferably no more than twice a day to achieve optimal benefit of the active ingredient.

In a preferred embodiment of the present invention, the hair styling composition is applied to wet hair and then air dried and/or dried with a hair dryer to achieve optimal hair styling benefits. If desired, the hair styling composition can be applied, for example, by hand and/or combed through the hair with a comb or brush.

The hair styling composition preferably maintains a hair style for a time period of at least 4 hours and more preferably for a time period of up to 18 hours. The hair styling composition has the advantage of treating dandruff and/or seborrheic dermatitis while maintaining a cosmetically acceptable hair style.

The hair styling compositions of the present invention may be prepared by any technique known to those skilled in the art. A preferred method of preparation is to disperse and/or dissolve the thickening agent(s) in water with mixing, and then to add the active ingredient and the desired hairstyling resin(s) to the thickened water mixture. The composition is then preferably adjusted to the desired pH.

### EXAMPLES

The following examples provide hair styling compositions useful in the present invention. The weight percentages listed in each of the following examples in Table 1 represent the amount of each ingredient on an active ingredient basis present in the hair styling composition.

The compositions were prepared by adding to water with mixing at a temperature of about 25° C. the thickening agent(s) (e.g., acrylic acid homopolymer, Acrylates/Vinyl Isodecanoate Crosspolymer, PVM/MA Decadiene Crosspolymer or Hydroxypropyl Guar). After the thickening agent(s) were completely hydrated, the zinc pyrithione was added and uniformly suspended in the aqueous mixture. After the zinc pyrithione was added, the hair styling resin(s) were added to the aqueous mixture with mixing. Following addition of the hair styling resin(s), the pH was adjusted to pH 6.

TABLE 1
Anti-dandruff hair styling compositions

| | Comp. | | | |
|---|---|---|---|---|
| | Example | Example | Example | Example |
| Ingredients | 1 | 2 | 3 | 4 |
| Water | qs to | qs to | qs to | qs to |
| | 100.00 | 100.00 | 100.00 | 100.00 |
| Acrylates/Vinyl | — | 0.35 | — | — |
| Isodecanoate Polymer¹ | | | | |
| PVM/MA Decadiene | — | — | 0.35 | — |
| Crosspolymer² | | | | |
| Hydroxypropyl Guar³ | — | — | — | 2.00 |
| Hydroxypropylcellulose⁴ | — | 0.50 | — | — |
| Hydroxyethylcellulose⁵ | — | — | 0.50 | — |
| polyacrylic acid | 0.5 | | | |
| homopolymer⁶ | | | | |
| Polyvinylpyrrolidone⁷ | 2.00 | 2.00 | | |
| Polyvinylpyrrolidone/ | — | — | 2.00 | |
| Vinyl Acetate⁸ | | | | |
| Polyquaternium-11⁹ | — | — | — | 1.00 |
| Triethanolamine | qs to pH | qs to pH | — | — |
| | 6.0 | 6.0 | | |
| Aminomethyl propanol | — | — | qs to pH | — |
| | | | 6.0 | |
| Sodium Hydroxymethyl- | — | — | — | qs to pH |
| glycinate | | | | 6.0 |
| Zinc pyrithione | | 0.11 | 0.11 | 0.11 |

¹ Stabylen 30, supplied by 3V, Inc.
² Stabileze QM, supplied by ISP
³ Jaguar HP 60, supplied by Rhodia
⁴ Klucel HS, supplied by Aqualon
⁵ Natrosol 250 HHR, supplied by Aqualon
⁶ Carbomer 940, supplied by BF Goodrich
⁷ Mw of about 630,000 daltons
⁸ PVP/VA W735, supplied by ISP
⁹ Quaternized polymer of vinyl pyrrolidone and dimethylaminoethyl methacrylate

The compositions of Examples 2 to 4 were gels having a pH of about 6.0 and a viscosity of about 5000 centipoises to about 20,000 centipoise. The compositions of Examples 2 to 4 exhibited excellent storage stability, showing no phase separation or ingredient precipitation after storage for several weeks at about temperatures of 4° C., about 25° C., and about 45° C. In contrast, Comparative Example 1 is an example of the use of a thickener that was unsuitable in that the thickening properties of the thickener deteriorated resulting in the active ingredient settling out of the composition.

Other hair styling compositions of the present invention can be prepared by varying the identity of the ingredients included in the compositions of Examples 2-4.

Although the present invention has been described above with respect to particular preferred embodiments, it will be apparent to those skilled in the art that numerous modifications and variations can be made to the present invention without departing from the scope of the present invention.

What is claimed is:

1. A hair styling composition comprising:
a) at least one active ingredient for the treatment of dandruff, seborrheic dermatitis or combinations thereof comprising a pyridinethione metal salt, octopirox, selenium disulfide, or combinations thereof;
b) at least about 0.01 weight percent of at least one polymeric thickening agent, wherein the thickening agent comprises a hydroxy C₁ to C₆ alkyl ether polyaldose, a copolymer having polymerized units of at least one carboxylic acid containing monomer and at least one hydrophobic monomer, or combinations thereof; and
c) at least about 0.1 weight percent of at least one hair styling resin; wherein the composition has a viscosity of at least 4,000 centipoises.

2. The composition of claim 1 wherein the composition is in the form of a gel and is aqueous-based.

3. The composition of claim 2 wherein the active ingredient for the treatment of dandruff or seborrheic dermatitis comprises a water insoluble salt of pyridinethione.

4. The composition of claim 3 wherein the water insoluble salt of pyridinethione comprises zinc pyrithione.

5. The composition of claim 3 wherein the thickening agent is water soluble or water swellable and comprises the polyaldose selected from a hydroxy C₁ to C₆ alkyl cellulose, or a hydroxy C₁ to C₆ alkyl guar, or combinations thereof.

6. The composition of claim 3 wherein the thickening agent is water soluble or water swellable and comprises the copolymer, wherein the carboxylic acid containing monomer is selected from (meth)acrylic acid, crotonic acid, maleic acid, maleic anhydride, salts of the foregoing monomers, or combinations thereof; the hydrophobic monomer is selected from vinyl esters of C₆ to C₁₈ carboxylic acids, C₁ to C₅ alkyl vinyl ethers, C₁ to C₈ alkyl esters of (meth)acrylic acid, or combinations thereof; and the copolymer is crosslinked.

7. The composition of claim 3 wherein the thickening agent is water soluble or water swellable and comprises a crosslinked copolymer having polymerized units of (meth)acrylic acid and a vinyl ester of a C₆ to C₁₈ carboxylic acid, a crosslinked copolymer having polymerized units of maleic anhydride or maleic acid and a C₁ to C₂ alkyl vinyl ether monomer, a hydroxy C₂ to C₃ alkyl cellulose, a hydroxy C₂ to C₃ alkyl guar, salts of the foregoing polymers, or combinations thereof.

8. The composition of claim 7 wherein the hair styling resin comprises at least one nonionic polymer, at least one amphoteric polymer, or combinations thereof.

9. The composition of claim 8 wherein the hair styling resin comprises homopolymers or copolymers of vinylpyrrolidone, copolymers having polymerized units of (meth)acryloyl C₁ to C₄ alkyl betaine monomer, homopolymers or copolymers having polymerized units of vinylcaprolactam, Corn Starch Modified, or combinations thereof.

10. The composition of claim 9 wherein the thickening agent comprises a crosslinked polymer having polymerized units of acrylic acid and vinyl isodecanoate, a crosslinked copolymer having polymerized units of maleic acid, vinyl methyl ether and decadiene, hydroxyethylcellulose, hydroxypropylcellulose, hydroxypropyl guar; salts thereof, or combinations thereof.

11. The composition of claim 10 wherein the hair styling resin comprises polyvinylpyrrolidone, a copolymer having polymerized units of vinyl acetate and vinylpyrrolidone, a copolymer having polymerized units of dimethylaminopropylmethacrylamide and vinyl pyrrolidone, a quaternized copolymer having polymerized units of vinyl pyrrolidone and dimethylaminoethyl methacrylate, a copolymer having polymerized units of (meth)acryloyl ethyl betaine and (meth)acrylic acid or salts thereof, polyvinylcaprolactam, Corn Starch Modified, or combinations thereof.

12. An aqueous-based hair styling composition comprising:
a) at least one active ingredient for the treatment of dandruff or seborrheic dermatitis comprising zinc pyrithione;
b) at least about 0.01 weight percent of at least one water soluble or water swellable polymeric thickening agent comprising a crosslinked copolymer having polymerized units of acrylic acid and vinyl isodecanoate or salts thereof, a crosslinked copolymer having polymerized units of maleic acid and vinyl methyl ether or salts thereof, hydroxypropyl guar, or combinations thereof, and
c) at least about 0.1 weight percent of at least one hair styling resin; wherein the composition has a viscosity ranging from about 4,000 centipoises to about 100,000 centipoises.

13. A method of treating a scalp or hair comprising applying to the hair or scalp a composition comprising
a) at least one active ingredient for the treatment of dandruff, seborrheic dermatitis or combinations thereof comprising a pyridinethione metal salt, octopirox, selenium disulfide, or combinations thereof;
b) at least about 0.01 weight percent of at least one polymeric thickening agent, wherein the thickening agent comprises a hydroxy C₁ to C₆ alkyl ether polyaldose, a copolymer having polymerized units of at least one carboxylic acid containing monomer and at least one hydrophobic monomer, or combinations thereof; and
c) at least about 0.1 weight percent of at least one hair styling resin; wherein the composition has a viscosity of at least about 4,000 centipoises; wherein the composition treats the scalp or hair for dandruff or seborrheic dermatitis and is capable of assisting in styling the hair or maintaining the hair in a hair style.

14. The method of claim 13 wherein the composition is applied to wet hair and the hair is styled after the application.

15. The method of claim 13 wherein the composition is in the form of a gel and is aqueous based.

16. The method of claim 15 wherein the active ingredient for the treatment of dandruff or seborrheic dermatitis comprises a water insoluble salt of pyridinethione.

17. The method of claim 16 wherein the thickening agent is water soluble or water swellable and comprises a crosslinked copolymer having polymerized units of (meth)acrylic acid and a vinyl ester of a C₆ to C₁₈ carboxylic acid, a crosslinked copolymer having polymerized units of maleic anhydride or maleic acid and a C₁ to C₂ alkyl vinyl ether monomer, a hydroxy C₂ to C₃ alkyl cellulose, a hydroxy C₂ to C₃ alkyl guar, salts of the foregoing polymers, or combinations thereof.

18. The method of claim 17 wherein the hair styling resin comprises homopolymers or copolymers of vinylpyrrolidone, copolymers having polymerized units of (meth)acryloyl C₁ to C₄ alkyl betaine monomer, homopolymers or copolymers having polymerized units of vinylcaprolactam, Corn Starch Modified, or combinations thereof.

19. The method of claim 18 wherein the thickening agent comprises a crosslinked polymer having polymerized units of acrylic acid and vinyl isodecanoate, a crosslinked copolymer having polymerized units of maleic acid, vinyl methyl ether and decadiene, hydroxyethylcellulose, hydroxypropylcellulose, hydroxypropyl guar, salts thereof, or combinations thereof.

20. The method of claim 19 wherein the hair styling resin comprises polyvinylpyrrolidone, a copolymer having polymerized units of vinyl acetate and vinylpyrrolidone, a copolymer having polymerized units of dimethylaminopropylmethacrylamide and vinyl pyrrolidone, a quaternized copolymer having polymerized units of vinyl pyrrolidone and dimethylaminoethyl methacrylate, a copolymer having polymerized units of (meth)acryloyl ethyl betaine and (meth)acrylic acid or salts thereof; polyvinylcaprolactam, Corn Starch Modified, or combinations thereof.
